(19) United States
(12) Patent Application Publication
(10) Pub. No.: US 2026/0286607 A1
(43) Pub. Date: Sep. 24, 2026

(54) DISCHARGE SCREW ARRANGEMENT

(71) Applicant: VALMET AB, Sundsvall (SE)

(72) Inventors: Örjan AHLGREN, Sundsvall (SE); Francois LAMBERT, Sundsvall (SE)

(73) Assignee: VALMET AB, Sundsvall (SE)

(21) Appl. No.: 19/476,063

(22) PCT Filed: Apr. 16, 2024
(86) PCT No.: PCT/SE2024/050360

§ 371 Date: Oct. 16, 2025

(30) Foreign Application Priority Data
May 3, 2023 (SE) 2330196-3

(51) Int. Cl. D21C 7/08

(52) U.S. Cl. CPC D21C 7/08

(57) ABSTRACT

A discharge arrangement for discharging lignocellulosic material includes: a screw conveyor section comprising an inlet for receiving lignocellulosic material from a treatment reactor; an outlet section configured to receive lignocellulosic material from the screw conveyor section; a conveyor screw rotatably arranged within the screw conveyor section for transporting the material in a material transport direction from the inlet to the outlet section; a material spreader rotatably arranged in the outlet section, which material spreader is rotatable in at least a first rotational direction around a rotational axis extending substantially in the material transport direction the material spreader; and a drive unit configured to rotate the material spreader around the rotational axis in at least said first rotational direction.

### TECHNICAL FIELD

The invention relates to the field of treatment of biomass material such as lignocellulosic material, and more specifically to discharge screw arrangements for discharging biomass from a biomass treatment reactor through an outlet, which outlet may be arranged for steam explosion discharge.

### BACKGROUND

Processes for treatment of biomass such as pre-treatment or more specifically pre-hydrolysis of biomass material such as lignocellulosic biomass material are known in the art. Pre-hydrolysis, which may also be referred to as hydrothermal treatment, is carried out at milder conditions compared to a hydrolysis process and is used for hydrolyzing the hemicellulose content of the biomass. The pre-hydrolysis process may be performed with added acid catalyst or without added catalyst (so-called autohydrolysis). The pre-hydrolysis process is used for example as a pretreatment stage in the pre-hydrolysis Kraft process (PHK), in processes for producing pellets, and in processes for producing ethanol as a pretreatment stage preceding a hydrolysis stage for hydrolyzing cellulose to sugar for subsequent fermentation to ethanol.

The pre-hydrolysis/hydrothermal treatment is performed at elevated pressure and temperature by adding the biomass to a pressurized reactor into which steam is added for heating the biomass to saturation temperature by direct condensation. The pressure level of the reactor which may be designed for continuous operation is in the range 5-30 bar and the retention time is in the range 1-20 minutes. The reactor may be horizontally or vertically arranged. In a typical horizontal reactor, an internal conveyor screw transports the biomass material through the reactor. The retention time can be controlled using the rotational speed of the screw. In a vertical reactor, a conveyor screw is typically arranged at a lower portion of the reactor for feeding the biomass material out from the reactor. In horizontal as well as vertical reactors, steam is added at one or more positions.

After treatment in the reactor, the lignocellulosic material is discharged from the reactor for further transport to subsequent processing equipment. Advantageously, the treated biomass is discharged (blown) from the reactor through a blow pipe/valve, so called steam explosion discharge. Steam explosion refers to a process step where the material undergoes a rapid/instantaneous pressure decrease. Thus, the hot and softened biomass from the thermal treatment is released or blown from the reactor through a blow valve or orifice, while the pressure drops to an environment with substantially lower pressure, such as below 5 bar, or preferably to substantially atmospheric pressure.

Discharging of the biomass material from the reactor may be carried out using a discharge screw arrangement. SE542682C2 discloses a discharge screw arrangement for discharging lignocellulosic material from a reactor, which discharge screw arrangement comprises a discharge screw rotatably arranged in a feeder pipe to transport the lignocellulosic material through the feeder pipe to an end of the feeder pipe where a material spreader is rotatably arranged to transport the lignocellulosic material towards a peripherally arranged outlet nozzle. Steam flows through the feeder pipe to help transport lignocellulosic material out through the outlet nozzle.

Although reference has been made above specifically to the pre-hydrolysis process, it is noted that the present invention relates to discharging of biomass material from a reactor regardless of the process carried out therein.

### SUMMARY

An object of the invention is to provide a discharge screw arrangement and a biomass treatment arrangement comprising such a discharge screw arrangement which provides improved discharge performance. More specifically, an object of the invention is to reduce deposits in the discharge screw arrangement and/or reduce steam consumption and/or to achieve a more stable discharge.

These and other objects are achieved by the present invention by means of a discharge screw arrangement, a discharge system, a treatment system and a method for controlling a discharge arrangement as defined in the appended claims.

According to a first aspect of the invention, there is provided a discharge arrangement for discharging biomass material such as lignocellulosic material. The discharge arrangement comprises a screw conveyor section provided with an inlet for receiving lignocellulosic material from a treatment reactor, and an outlet section arranged to receive lignocellulosic material from the screw conveyor section. A conveyor screw is rotatably arranged within the screw conveyor section for transporting the material in a material transport direction from the inlet to the outlet section. A material spreader is rotatably arranged in the outlet section, which material spreader is rotatable in at least a first rotational direction around a rotational axis extending substantially in the material transport direction the material spreader. The discharge arrangement can comprise a drive unit (such as an electric motor) configured to rotate the material spreader around the rotational axis in at least said first rotational direction. A first opening and a second opening are arranged peripherally of the material spreader in a side wall of the outlet section, said first and second openings being arranged at a distance from one another along an inner perimeter of the side wall. The material spreader is configured to, during rotation of the material spreader in said first rotational direction, transport the lignocellulosic material in the first rotational direction and radially outwards towards the first opening for discharge of the lignocellulosic material from the outlet section. The second opening is connected to a steam line connectable to a steam source to provide discharge steam into the outlet section.

The screw conveyor section may be described as elongated, i.e. having a substantially greater length (in a longitudinal direction being parallel with the rotational axis) than width or diameter. The screw conveyor section may be substantially cylindrically formed or tubular, and/or may be described as pipe shaped. The discharge arrangement can be substantially horizontally arranged, i.e. with its longitudinal direction and/or the rotational axis in a substantially horizontal plane. The inlet can be arranged at a first longitudinal end (an upstream end as seen in the flow direction) of the screw conveyor section and/or at an upper portion thereof. In embodiments where the biomass treatment reactor is substantially horizontally arranged, that the biomass material can fall into the inlet into the screw conveyor section by means of gravity.

The outlet section may be described as substantially tubular or cylindrical. The side wall may be described as the mantle or lateral surface of the outlet section. The first and second openings may be described as arranged at a distance from one another, or spaced apart, in the peripheral or circumferential direction of the outlet section. The first and/or second openings can comprise outlet nozzle(s) in the side wall of the outlet section and can have a fixed or adjustable size. The first and second openings can be located at any position along the inner perimeter/circumference of the outlet section side wall.

The invention is based on the insight that a more stable discharge and/or reduced steam consumption and/or reduced deposits can be achieved by adding the steam to the outlet section (preferably in such a manner that it propels the biomass material towards the openings/outlets) rather than adding the steam needed for discharge upstream such as in the treatment reactor or in the screw conveyor section. A more stable discharge and reduced steam consumption can be achieved since the discharge can be accurately controlled by means of adjusting the steam addition to the outlet section. Reduced deposits may be achieved since the steam added in the outlet section may have a cleaning effect on the material spreader and/or on the discharge opening.

In embodiments, the conveyor screw is arranged to rotate in the first rotational direction, i.e. the conveyor screw and the material spreader may rotate in the same direction. This may provide advantageous discharge performance.

In embodiments, the material spreader is further rotatable in a second rotational direction being opposite the first rotational direction. The drive unit can further be configured to rotate the material spreader in said second rotational direction. The material spreader can be configured to, during rotation of the material spreader in said second rotational direction, transport the lignocellulosic material in the second rotational direction and radially outwards towards the first and/or second opening for discharge of the lignocellulosic material from the outlet section. In embodiments where the material spreader is configured to, during rotation of the material spreader in said second rotational direction, transport the lignocellulosic material towards the second opening, the first opening can be connected to an additional steam line connectable to said steam source (or to an additional steam source being different than the steam source) to provide discharge steam into the outlet section via the first opening. It is understood that when the material spreader is rotated in the second rotational direction to transport the lignocellulosic material towards the second opening, steam supply to the second opening from the steam line is turned off, for example by closing a control valve. Instead, steam may be supplied to the outlet section from the additional steam line via the first opening or via other steam inlet(s). Correspondingly, when the material spreader is rotated (in the first or second rotational direction) to transport the lignocellulosic material towards the first opening, steam supply to the first opening from the (optional) additional steam line is turned off, for example by closing a control valve.

In embodiments, the first opening and/or second opening is/are directed substantially parallel with a tangent to the first or second rotational direction of the material spreader. The first and/or second openings may alternatively be described as directed substantially parallel with a tangential direction of the side wall of the outlet section (which outlet section in such an embodiment may be described as substantially tubular/cylindrical). Openings(s) directed tangentially ensures that the material is propelled by the material spreader into the first opening and/or second opening.

Herein, the tangent to a rotational direction of the material spreader is the forward pointing tangent or tangent vector to the travelling path of the outermost portion of the material spreader (such as a tip of a blade of the material spreader) being rotated in the rotational direction of the material spreader. The tangent may be drawn from any point on the travelling path of the outermost portion of the material spreader.

Alternatively, the first opening and/or the second opening can be directed substantially radially relative the rotational axis of the material spreader. Alternatively, the first opening and/or the second opening can be directed in any direction between radially and parallel with the above-described tangential direction.

In embodiments, the first and second openings are directed in opposite directions.

In embodiments, the material spreader is provided with at least one radially extending blade configured to rotate around the rotational axis to transport the lignocellulosic material radially outwards and in the first or second rotational direction rotation during rotation of the material spreader (depending on direction of rotation). One or more blades, or each blade, of the material spreader can be connected to or integrally formed with a rotatable disc arranged to be rotated by the drive unit around the rotational axis of the material spreader. For example, the rotatable disc can have a diameter corresponding to the radial extension of the blades, wherein the blades are connected to or is integrally formed with the disc along their radial extension. Preferably, the material spreader comprises at least two blades, and even more preferably three or four blades. One or more, or each, of the at least one radially extending blade comprises at least one surface portion being curved in a direction opposite of the first (or second) rotational direction, or is curved as a whole in a direction opposite of the first (or second) rotational direction. Such embodiments are advantageous since the curved shape facilitates transport in the radial direction when the material spreader rotates in the first (or second) rotational direction.

In embodiments, the discharge arrangement further comprises one or more discharge valves arranged to regulate the flow of lignocellulosic material through the first opening and/or through the second opening. A discharge valve of the one or more discharge valves can be arranged in the first opening and/or in the second opening. The material spreader can be arranged with the at least one blade to rotate with the radially outermost portion thereof (such as a tip of the blade) at a distance of less than 40 mm, such as less than 20 mm, or less than 5 mm from at least one of the one or more discharge valves. Such embodiments are advantageous since the blades rotating close to the discharge valve(s) serve as cleaning means reducing the risk of clogging the discharge valve(s).

In embodiments, the first opening and/or the second opening is provided with one or more nozzles to restrict a flow of lignocellulosic material therethrough. For example, a nozzle can be arranged in the first and/or second openings. The material spreader can be arranged with the at least one blade to rotate with the radially outermost portion thereof (such as a tip of the blade) at a distance of less than 40 mm, such as less than 20 mm, or less than 5 mm from at least one of the nozzle(s). Such embodiments are advantageous since the blades rotating close to the nozzle(s) serve as cleaning means reducing the risk of clogging the nozzle(s). It is understood that any flow of steam provided to the first and/or second opening (from the steam line and/or additional steam line) may pass through the respective nozzle(s). Alternatively, the steam line/additional steam line may be connected to the first/second opening to bypass the nozzle(s).

In embodiments, the side wall of the outlet section is provided with at least one steam inlet connected or connectable to said steam source, each steam inlet being arranged at a distance from the first and second openings (and from other steam inlets, if any) along an inner perimeter of the side wall. In other words, each steam inlet is arranged at a distance from the first and second openings (and from other steam inlets, if any) in circumferential direction of the outlet section. The steam inlets can be distributed symmetrically and/or equidistantly (as seen in the circumferential direction) around the outlet section.

In embodiments, each steam inlet is directed at an angle relative the first rotational direction which lies within an interval of 0 to 90 degrees, i.e. forms an angle of 0 to 90 degrees relative the first rotational direction. In other words, the steam inlets are arranged to inject steam in a direction being substantially perpendicular with the first rotational direction (90 degrees angle), or in the first rotational direction (0 degrees angle), or at any angle therebetween. Injecting steam into the outlet section at such angles may be advantageous since it may facilitates discharging of the lignocellulosic material and/or may reduce the risk of clogging.

In embodiments, the steam line and/or the additional steam line is (each) provided with a control valve and/or at least one (or each) steam inlet is provided with a control valve. This may be advantageous since the steam flow to the outlet section may be controlled/optimized.

In embodiments, the discharge screw arrangement further comprises at least one steam explosion device connected to the first and/or second opening. Steam explosion discharge devices are known in the art and are configured to discharge/blow the material while rapidly decreasing the pressure, for instance to ambient pressure. The steam explosion discharge device can comprise a blow pipe and/or a blow valve and/or a blow orifice.

According to a second aspect of the invention, there is provided a discharge system comprising a discharge arrangement according to the first aspect of the invention or an embodiment thereof and a control system, wherein the steam line and the steam inlets are provided with respective control valves. The control system is configured to, during rotation of the material spreader in the first rotational direction, control the control valves such that at least 50%, or at least 80% of an overall steam flow is provided to the second opening via said steam line (and the rest of the steam flow is provided to the steam inlets). In embodiments where the material spreader is further rotatable in a second rotational direction and the additional steam line is provided with a control valve, the control system is further configured to, during rotation of the material spreader in the second rotational direction, control the control valves such that at least 50%, or at least 80% of an overall steam flow is provided to the first opening via said additional steam line (and the rest of the steam flow is provided to the steam inlets).

According to a third aspect of the invention, there is provided a treatment arrangement comprising a treatment reactor for treating lignocellulosic material and a discharge screw arrangement according to the first aspect of the invention or an embodiment thereof, wherein the inlet of the discharge screw arrangement is arranged to receive lignocellulosic material from an outlet of the treatment reactor. The treatment reactor can be a pre-hydrolysis reactor.

According to a fourth aspect of the invention, there is provided a method for controlling a discharge arrangement in which the steam line and the steam inlets are provided with respective control valves. The method comprises, during rotation of the material spreader in the first rotational direction, controlling the control valves of the steam line and of the steam inlets such that at least 50%, or at least 80% of an overall steam flow is provided to the second opening via said steam line (and the rest of the steam flow is provided to the steam inlets). In embodiments where the material spreader is further rotatable in a second rotational direction and the additional steam line is provided with a control valve, the method further comprises, during rotation of the material spreader in the second rotational direction, controlling the control valves such that at least 50%, or at least 80% of an overall steam flow is provided to the first opening via said additional steam line (and the rest of the steam flow is provided to the steam inlets).

According to a fifth aspect of the invention, there is provided a treatment system corresponding to the discharge arrangement according to the first aspect of the invention (or corresponding to the discharge system according to the second aspect of the invention) except that the screw conveyor section is replaced with a substantially horizontally arranged treatment reactor vessel/chamber provided with an inlet for receiving lignocellulosic material, wherein a conveyor screw is rotatably arranged within the reactor vessel for transporting the lignocellulosic material in a material transport direction from the inlet to the outlet section. The reactor vessel can be configured for hydrothermal treatment/pre-hydrolysis of the lignocellulosic material, for example by being provided with one or more steam inlets for providing steam to heat the lignocellulosic material.

The features of the embodiments described above are combinable in any practically realizable way to form embodiments having combinations of these features. Further, all features and advantages of embodiments described above with reference to the first aspect of the invention can be applied in corresponding embodiments of the system according to the second, third, fourth and fifth aspects of the invention and vice versa. In particular, it is noted that the above-described features of embodiments of the first and second aspects of the invention can be applied in corresponding embodiments of the system according to the fifth aspect of the invention, where it is understood that the reactor vessel/chamber corresponds to the screw conveyor section.

### BRIEF DESCRIPTION OF THE DRAWINGS

Above discussed and other aspects of the present invention will now be described in more detail using the appended drawings, which show presently preferred embodiments of the invention, wherein:

FIG. 1 shows a schematic top view of an embodiment of a discharge system according to the second aspect of the invention, which discharge system comprises a discharge arrangement according to the first aspect of the invention,

FIG. 2 shows a schematic side view of an embodiment of a treatment arrangement according to the third aspect of the invention,

FIG. 3 shows a cross-section view of the outlet section of the discharge arrangement in FIG. 1, the cross-section being taken in a plane being perpendicular to the rotational axis of the material spreader,

FIG. 4 shows a cross-section view of the outlet section of another embodiment of a discharge arrangement according to the first aspect of the invention, and

FIG. 5 shows a cross-section view of the outlet section of yet another embodiment of a discharge arrangement according to the first aspect of the invention.

### DETAILED DESCRIPTION

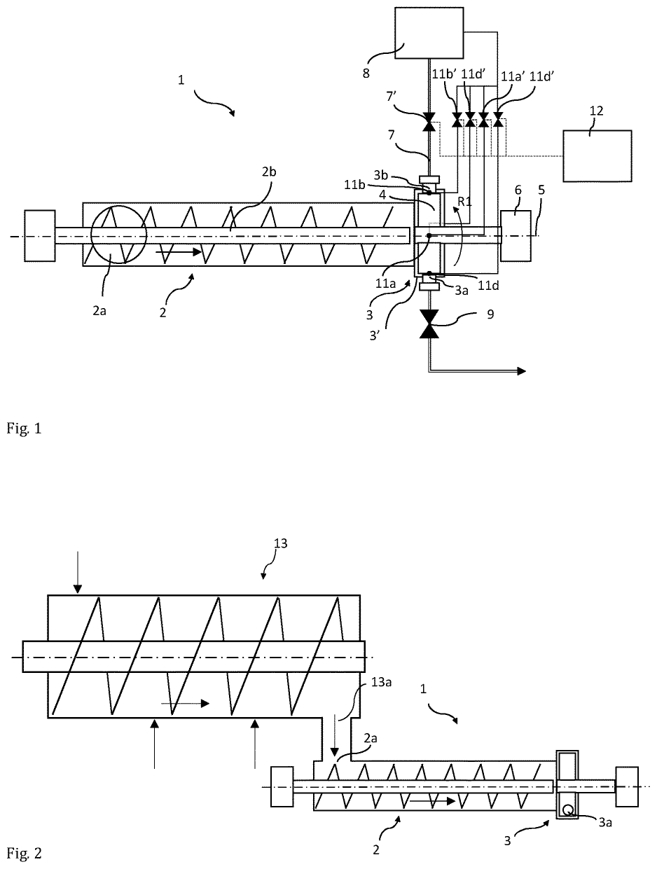

FIG. 1 shows a schematic top view of an embodiment of a discharge system according to the second aspect of the invention, which discharge system comprises a discharge arrangement 1 according to the first aspect of the invention.

The discharge arrangement comprise a screw conveyor section 2 provided with an inlet 2a for receiving lignocellulosic material from a treatment reactor, an outlet section 3 arranged to receive lignocellulosic material from the screw conveyor section 2. The discharge arrangement is illustrated with the outer surfaces/housings of the sections 2 and 3 transparent to show the parts therein. In this embodiment, the screw conveyor section and the outlet section are formed as separate units/parts connected to each other, but it is also foreseeable that they are formed as an integral part. The screw conveyor section 2 is substantially cylindrically formed and elongated, i.e. having a substantially greater length (in the material transport direction) than width or diameter. The discharge arrangement is substantially horizontally arranged, i.e. with its material transport direction/rotational axis in a substantially horizontal plane.

A conveyor screw 2b is rotatably arranged within the screw conveyor section 2 for transporting the material in a material transport direction (as indicated by the arrow in 2) from the inlet 2a to the outlet section 3. The inlet 2a is arranged at a first longitudinal end (to the left in the figure) of the screw conveyor section 2 at an upper portion thereof.

A material spreader 4 is rotatably arranged in the outlet section 3, which material spreader is rotatable in a first rotational direction R1 around a rotational axis extending 5 substantially in the material transport direction the material spreader. In this embodiment the rotational axis of the material spreader 4 and the conveyor screw 2b coincide.

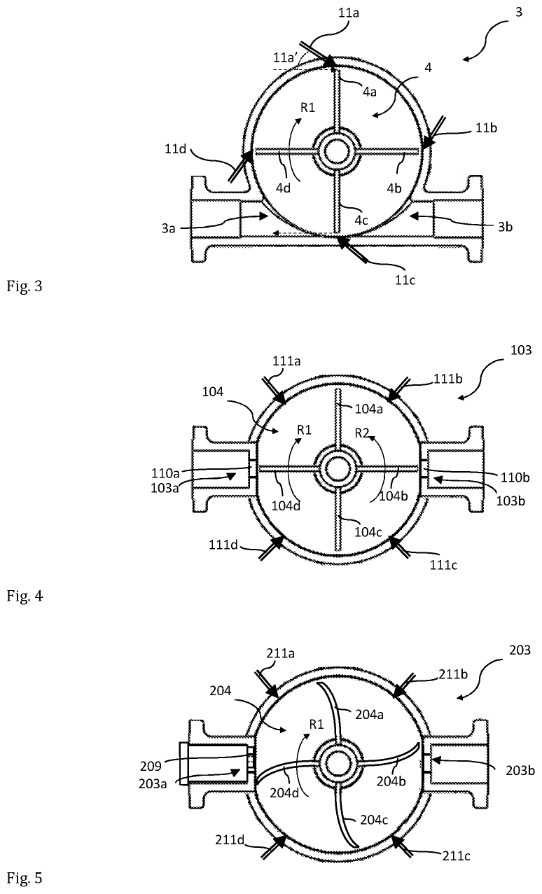

A drive unit (such as an electric motor) 6 is configured to rotate the material spreader 4 around the rotational axis in the first rotational direction R1. A first opening 3a and a second opening 3b are arranged peripherally of the material spreader 4 in a side wall 3′ being the mantle surface of the substantially cylindrical outlet section 3, the first and second openings being arranged at a distance from one another along an inner perimeter of the side wall, i.e. at a distance from each other in the circumferential direction of the outlet section. The first and second openings are arranged at a bottom portion of the outlet section (see FIG. 3).

The material spreader 4 is configured to, during rotation of the material spreader in the first rotational direction R1, transport the lignocellulosic material in the first rotational direction and radially outwards towards the first opening 3a for discharge of the lignocellulosic material from the outlet section 3. The second opening 3b is connected to a steam line 7 connected to a steam source 8 to provide discharge steam into the outlet section 3.

A steam explosion device comprising a blow line provided with a blow/discharge valve 9 is connected to the first opening to regulate the flow of lignocellulosic material through the first opening to achieve rapid pressure decrease/steam explosion discharge.

In addition to steam being provided to the outlet section via the second opening, the side wall 3′ of the outlet section 3 is provided with four steam inlets 11a-d (11c is visible in FIG. 3) distributed around the circumference of the outlet section, the steam inlets being connectable to the steam source 8 via a respective control valve 11a′-d′.

The steam line 7 is also provided with a control valve 7′. A control system 12 is configured to control the control valves 7′, 11a′-d′ of the steam line and of the steam inlets such that at least 50 %, or at least 80% of an overall steam flow is provided to the second opening 3b via said steam line 7.

FIG. 2 shows a schematic side view of an embodiment of a treatment arrangement according to the third aspect of the invention. The treatment arrangement comprises a biomass treatment reactor 13 formed by a horizontally arranged cylindrical reactor vessel provided with an internal conveyor screw rotatably arranged in the reactor vessel. The internal conveyor screw is configured to mechanically transport the biomass material through the reactor vessel in a conveying direction (indicated by the arrow in the reactor vessel) in parallel with the rotational axis of the internal conveyor screw and out through an outlet 13a, the outlet being disposed at a downstream end of the reactor vessel. The reactor vessel is provided with an inlet (schematically indicated by an arrow) for lignocellulosic biomass at a flow-wise upstream end of the reactor vessel at an upper portion of the reactor vessel. The reactor vessel is further provided with steam injection nozzles (schematically indicated by two arrows at the bottom of the reactor vessel) for hydrothermal treatment of the biomass.

The biomass treatment arrangement further comprises the discharge screw arrangement 1 shown in FIG. 1 and described above. The inlet 2a of the screw conveyor section 2 is arranged to receive biomass material from the outlet 13a of the treatment reactor. The discharge screw arrangement is located vertically below the reactor vessel at the downstream end of the reactor vessel, such that the biomass material falls down vertically from the outlet 13a of the reactor vessel into the inlet 2a of the discharge screw arrangement.

Unlike in FIG. 1, the discharge screw arrangement 1 is seen in a side view with the first opening 3a facing outwards and the second opening 3b not visible. In FIG. 2, the treatment arrangement is illustrated with the outer surfaces/housings of 2, 3 and 13 transparent to show the parts therein.

FIG. 3 shows a cross-section view of the outlet section of the discharge arrangement in FIG. 1, the cross-section being taken in a plane being perpendicular to the rotational axis of the material spreader. The first opening 3a is directed substantially along a tangent to the first rotational direction R1 of the material spreader 4 (which also coincides with the tangential direction of the outlet section 3. The tangential direction is indicated by the dotted arrow below 4c. The second opening 3b is directed in the opposite direction to the first opening 3a.

As can be seen in FIG. 3, the material spreader 4 is provided with four radially extending blades 4a-d rotating around the rotational axis of the material spreader to transport the lignocellulosic material. The blades are arranged at 90 degrees interval from each other. The blades are substantially plane, i.e. extend in a respective radial plane.

The steam inlets 11a-d are directed at an angle (11a′ for example) of about 45 degrees relative the tangent to the first rotational direction/the outlet section. As can be seen in FIG. 3, the steam inlets are arranged at a circumferential distance from each other and from the first and second openings 3a-b.

FIG. 4 shows a cross-section view of the outlet section 103 of another embodiment of a discharge arrangement according to the first aspect of the invention, the cross-section being taken in a plane being perpendicular to the rotational axis of the material spreader 104. The material spreader 104 comprises blades 104a-d and corresponds to the material spreader 4 in FIG. 3. The embodiment in FIG. 4 differs from the embodiment in FIG. 3 in that the first and second openings 103a-b are directed substantially radially (in opposite directions) relative the rotational axis. Further, the first opening and second openings are provided with a respective nozzle 110a-b to restrict a flow of lignocellulosic material therethrough, the nozzles having a fixed flow area. Although the openings are provided with a nozzle, it is foreseeable that an (external) blow/discharge valve (corresponding to ref. 9 in FIG. 1) is connected thereto. The material spreader 104 is arranged to rotate with a radially outermost portion (the tips of the blades 104a-d) at a distance of less than 5 mm from each of the nozzles 110a-b. The outlet section is provided with four steam inlets arranged at a circumferential distance from each other and from the first and second openings 103a-b, the steam inlets being directed at 90 degrees angle relative the tangential direction, i.e. radially. A further difference is that the material spreader is configured to rotate interchangeably in the first rotational direction R1 or in the second rotational direction R2.

The material spreader may be configured to, during rotation of the material spreader in the second rotational direction R2, transport the lignocellulosic material in the second rotational direction and radially outwards towards the first opening 103a and/or towards the second opening 103b for discharge of the lignocellulosic material from the outlet section.

The first opening 103a may be connected to an additional steam line connectable to the steam source (or to an additional steam source being different than the steam source) to provide discharge steam into the outlet section via the first opening.

The material spreader may operate as follows:
When lignocellulosic material is to be discharged through the first opening 103a (for example by means of rotation in direction R1), steam is supplied via a steam line (corresponding to 7 in FIG. 1) to the second opening 103b and/or via steam inlets 111a-d. If an additional steam line connected to the first opening 103a is present, the supply of steam to the first opening 103a is turned off by means of a control valve.
When lignocellulosic material is to be discharged through the second opening 103b (by means of rotation in direction R2), the supply of steam thereto is turned off by means of a control valve (corresponding to 7′ in FIG. 1). If an additional steam line connected to the first opening 103a is present, steam is supplied to the first opening 103a and/or via steam inlets 111a-d. Otherwise, steam is supplied via steam inlets 111a-d only.
When lignocellulosic material is to be discharged through both openings 103a-b, steam is supplied via steam inlets 111a-d only.

The above-described operation may be achieved using a control system (corresponding to 12 in FIG. 1) controlling the control valve of the steam line connected to the second opening, the control valves of the steam inlets 111a-d, and if an additional steam line is present, the control valve connected thereto.

Other embodiments correspond to the embodiment in FIG. 4, with or without optional features as described above, except that the first and second openings are directed as shown in FIG. 3 (tangentially). In such embodiments (with tangentially directed openings) the material spreader is normally configured to, during rotation in the first rotational direction R1, transport the material towards the first opening, and during rotation in the second rotational direction R2, transport the material towards the second opening.

FIG. 5 shows a cross-section view of the outlet section 203 of yet another embodiment of a discharge arrangement according to the first aspect of the invention, the cross-section being taken in a plane being perpendicular to the rotational axis of the material spreader 204. The material spreader 204 comprises four blades 204a-d curved in the opposite direction to the first rotational direction R1 and is configured to rotate only in direction R1. The first and second openings 203a-b are directed substantially radially (in opposite directions) relative the rotational axis. A discharge valve 209 is arranged in the first opening 203a (the discharge valve is schematically illustrated with varying effective flow area indicated by the dotted lines). The material spreader 204 is arranged to rotate with a radially outermost portion (the tips of the blades 104a-d) at a distance of less than 5 mm from the discharge valve 209. The outlet section is provided with four steam inlets 211a-d corresponding to the steam inlets in FIG. 4.

The description above and the appended drawings are to be considered as non-limiting examples of the invention. The person skilled in the art realizes that several changes and modifications may be made within the scope of the invention. For example, the steam inlets can be arranged at a different angle (although preferably within an interval of 0 to 90 degrees relative to a tangential direction). Further, the number of steam inlets can be varied. Further, the outlet section in the embodiment in FIG. 1-2 can be replaced with the outlet section shown in FIG. 4-5 and described above. The steam inlets in such outlet sections can be controlled in a corresponding manner as described above with reference to FIG. 1.

1-17. (canceled)

18. A discharge arrangement for discharging lignocellulosic material, said discharge arrangement comprising:
a screw conveyor section comprising an inlet for receiving lignocellulosic material from a treatment reactor;
an outlet section configured to receive lignocellulosic material from the screw conveyor section;
a conveyor screw rotatably arranged within the screw conveyor section for transporting the material in a material transport direction from the inlet to the outlet section;
a material spreader rotatably arranged in the outlet section, which material spreader is rotatable in at least a first rotational direction around a rotational axis extending substantially in the material transport direction the material spreader;
a drive unit configured to rotate the material spreader around the rotational axis in at least said first rotational direction; wherein:
a first opening and a second opening are arranged peripherally of the material spreader in a side wall of the outlet section, said first and second openings being arranged at a distance from one another along an inner perimeter of the side wall,
the material spreader is configured to, during rotation of the material spreader in said first rotational direction, transport the lignocellulosic material in the first rotational direction and radially outwards towards the first opening for discharge of the lignocellulosic material from the outlet section,
the second opening is connected to a steam line connectable to a steam source to provide discharge steam into the outlet section, and
the material spreader is further rotatable in a second rotational direction being opposite the first rotational direction, and wherein the drive unit is further configured to rotate the material spreader in said second rotational direction, wherein the material spreader is configured to, during rotation of the material spreader in said second rotational direction, transport the lignocellulosic material in the second rotational direction and radially outwards towards the first opening and/or the second opening for discharge of the lignocellulosic material from the outlet section.

19. The discharge arrangement according to claim 18, wherein the material spreader is configured to, during rotation of the material spreader in said second rotational direction, transport the lignocellulosic material in the second rotational direction and radially outwards towards the second opening for discharge of the lignocellulosic material from the outlet section, and wherein the first opening is connected to an additional steam line connectable to said steam source to provide discharge steam into the outlet section via the first opening.

20. The discharge arrangement according to claim 18, wherein the first opening and/or the second opening is directed substantially parallel with a tangent to the first rotational direction of the material spreader.

21. The discharge arrangement according to claim 18, wherein the first opening and/or the second opening are directed substantially radially relative the rotational axis.

22. The discharge arrangement according to claim 18, wherein the first and second openings are directed in opposite directions.

23. The discharge arrangement according to claim 18, wherein said material spreader is provided with at least one radially extending blade configured to rotate around the rotational axis to transport the lignocellulosic material.

24. The discharge arrangement according to claim 23, wherein one or more of the at least one radially extending blade comprises at least one surface portion being curved in a direction opposite of the first rotational direction.

25. The discharge arrangement according to claim 18, further comprising one or more discharge valves arranged to regulate the flow of lignocellulosic material through the first opening and/or through the second opening.

26. The discharge arrangement according to claim 25, wherein a discharge valve of the one or more discharge valves is arranged in said first opening and/or in said second opening.

27. The discharge arrangement according to claim 23, wherein:
a discharge valve of the one or more discharge valves is arranged in said first opening and/or in said second opening, and
said material spreader is arranged to rotate with a radially outermost portion thereof at a distance of less than 40 mm, such as less than 20 mm, or less than 5 mm from at least one of the one or more discharge valves.

28. The discharge arrangement according to claim 24, wherein said first opening and/or said second opening is provided with one or more nozzles to restrict a flow of lignocellulosic material therethrough.

29. The discharge arrangement according to claim 23, wherein:
said first opening and/or said second opening is provided with one or more nozzles to restrict a flow of lignocellulosic material therethrough, and
said material spreader is arranged to rotate with a radially outermost portion thereof at a distance of less than 40 mm, such as less than 20 mm, or less than 5 mm from at least one of the one or more nozzles.

30. The discharge arrangement according to claim 18, wherein the side wall of the outlet section is provided with at least one steam inlet connectable to said steam source, each steam inlet being arranged at a distance from the first and second openings along an inner perimeter of the side wall.

31. The discharge arrangement according to claim 30, wherein each steam inlet is directed at an angle relative the first rotational direction which lies within an interval of 0 to 90 degrees.

32. The discharge arrangement according to claim 30, wherein said steam line is provided with a control valve and wherein each steam inlet is provided with a control valve.

33. A system comprising:
the discharge arrangement according to claim 32; and
a control system configured to control the control valves of the steam line and of the steam inlets such that at least 50% of an overall steam flow is provided to the second opening via said steam line.

34. A treatment arrangement comprising:
a treatment reactor for treating lignocellulosic material; and
a discharge screw arrangement according to claim 18, wherein:
the inlet of the discharge screw arrangement is arranged to receive lignocellulosic material from an outlet of the treatment reactor.
